(19) United States
(12) Patent Application Publication
(10) Pub. No.: US 2015/0139930 A1
(43) Pub. Date: May 21, 2015

(54) Personal Care Compositions Including Narrow Molecular Weight Range Copolymers

(71) Applicant: Akzo Nobel Chemicals International B.V., Amersfoort (NL)

(72) Inventors: Rama Subramanium Chandran, Bridgewater, NJ (US); Michael Timothy Philbin, Hopewell, NJ (US)

(73) Assignee: Akzo Nobel Chemicals International B.V., Amersfoort (NL)

(21) Appl. No.: 14/406,489

(22) PCT Filed: Jun. 11, 2013
(86) PCT No.: PCT/EP2013/061939

§ 371 Date: Dec. 8, 2014

Related U.S. Application Data
(60) Provisional application No. 61/659,464, filed on Jun. 14, 2012.

(30) Foreign Application Priority Data
Aug. 13, 2012 (EP) 12180223.5

(51) Int. Cl. A61K 8/81; A61Q 5/06; A61K 8/04

(52) U.S. Cl. CPC A61K 8/8158; A61K 8/8152; A61K 8/046; A61Q 5/06; A61K 2800/54
USPC 424/70.16

(57) ABSTRACT

Personal Care compositions including one or more narrow molecular weight range copolymers have certain weight average molecular weights that provide low viscosity profiles and improved spray performance while also providing improved shampoo removability, hold, stiffness and feel compared to un-fractionated copolymers.

### FIELD OF THE INVENTION

The present invention relates to polymers suitable for use in personal care compositions. More specifically, the present invention relates to personal care composition, such as hair styling compositions, comprising narrow molecular weight range copolymers that provide improved stiffness and feel based on a selection of one or more narrow molecular weight ranges of the copolymer.

### BACKGROUND

Hair styling fixative polymers have been around for many years to provide the user the ability to place the hair in a given style and to afford a long lasting hold. Typically, this has been achieved through higher molecular weight polymers to provide the desired stiffness and modulus.

Further, fixative polymers also need to be easily applied to the hair in a uniform and effective manner. This can be achieved by means such as sprays (aerosol or mechanical pump) and with gels. Gels are generally applied to the hair prior to setting, whereas the sprays may be applied to the hair after curlers are used. In order to produce suitable sprayability and uniform coverage for aerosol and pump type applicators, the viscosity of the formulation to be sprayed, and thus the viscosity of the fixative polymers, must also be controlled. If the viscosity is too high (e.g. greater than 16 cps), the formulation will be too thick to be sprayable.

In addition to achieving the desired stiffness, modulus and viscosity requirements, the hair fixative polymers must also be prepared so as to provide the requisite performance of the polymer in formulations with respect to subjective evaluations, such as feel, stiffness, humidity resistance and flaking.

Accordingly, there is a need to provide improved hair styling fixative polymers to provide hair fixative formulations that can maximize coverage and performance of the polymer with respect to feel, stiffness, humidity resistance and flaking in formulations while also balancing the desired viscosity requirements.

### SUMMARY OF THE INVENTION

It is now been found that by utilizing copolymers with narrow molecular weight ranges, certain narrow molecular weight copolymers provide surprisingly higher levels of stiffness and feel compared to conventional polymers having a wide range of molecular weight or polymers having higher molecular weights.

Accordingly, the present invention relates to a personal care composition comprising one or more narrow molecular weight range copolymers and one or more solvents, wherein the one or more narrow molecular weight range copolymers have a weight average molecular weight range chosen from about 70,000 Daltons or greater or about 55,000 Daltons or less.

### DETAILED DESCRIPTION OF THE INVENTION

As used herein, the recitations of numerical ranges by endpoints include all numbers subsumed within that range (e.g., 1 to 5 includes 1, 1.5, 2, 2.75, 3, 3.80, 4, 5, etc.). In addition, it is to be understood that for embodiments including ranges as described herein, the respective lower endpoints and respective upper endpoints described include combinations of the various lower and upper endpoints. For example, for ranges of 1 to 20 and 5 to 10, respectively, the ranges also include, without limitation, 1 to 10 and 5 to 20.

As is typical with polymerizations, the polymer that is formulated contains a distribution of molecular weights. The polydispersity index for a given polymer indicates the variation of molecular weights in a homo- or co-polymer. While variables, such as monomer concentration, initiator concentration, polymerization efficiency, chain termination kinetics and the like, can be manipulated to vary the polydisperisity index, it is well known that polymers generally have some variation in average molecular weight.

Contrary to conventional wisdom in the art that higher molecular weight provides increases in stiffness and modulus, it has now been found that copolymers with narrow molecular weight ranges, and lower molecular weight copolymers, surprisingly provide higher levels of stiffness and feel as compared to copolymers having wider ranges of molecular weights copolymer. In certain personal care applications, such as in hair styling, these narrow molecular weight range copolymers having certain molecular weight ranges provide properties that were normally associated with higher molecular weight fractions. Thus, stiffness and feel can be achieved using lower molecular weight copolymers, which then also have more desirable, lower viscosity profiles associated with them. This lower viscosity provides better spray patterns and more uniform spray coverage.

For purposes of the present invention, narrow molecular weight range copolymers are defined as those copolymers having a weight average molecular weight of about 70,000 to about 90,000 Daltons or from about 10,000 to about 55,000 Daltons. In an embodiment, the narrow molecular weight copolymer may have a weight average molecular weight of from about 75,000 to about 80,000 Daltons. In yet another embodiment, the narrow molecular weight range copolymers may have a weight average molecular weight from about 10,000 to about 55,000 Daltons. In yet another embodiment, the narrow molecular weight range copolymers have a weight average molecular weight in the range from about 40,000 to about 55,000 Daltons. In still yet another embodiment, the one or more narrow molecular weight range copolymers may have a weight average molecular weight from about 45,000 to about 55,000 Daltons.

The polymers of this invention can be further defined according to their the polydispersity index (PDI). PDI is defined as:

$$PDI = \frac{Mw}{Mn}$$

where Mw is the weight average molecular weight of the copolymer and Mn is the number average molecular weight of the copolymer. This ratio provides a measure of the variation of molecular weights of a given (co)polymer. For example, a polydispersity index of 1 means that all of the polymer chains have the same molecular weight. The higher the polydispersity index, the broader are the variations in molecular weights. Accordingly, in an embodiment of this invention, the polydispersity index will be less than about 4, in another embodiment preferably less than about 3, in another embodiment most preferably less than about 2. Also, as defined herein, broad molecular weight copolymers as those having a wide spectrum of molecular weights and polydispersity indexes of greater than about 4.

Typical styling products are applied to the hair by either a spray (pump or aerosol) or as a mousse or gel. Each application method has certain unique formulation requirements, such as the inclusion of a propellant in the case of an aerosol spray or the inclusion of a gelling agent in the case of mousse or gelled product. The narrow molecular weight range copolymer and/or copolymer fractions should be compatible with the other ingredients to minimize or prevent the narrow molecular weight range copolymer fractions from precipitating or hazing out of solution. This can be managed by varying the amount and type of other monomers in the styling polymer. For example, hydrophobic monomers, such as (meth)acrylates, contribute to the compatibility with propellants and non-aqueous solvents, whereas anionic and cationic monomers will tend to make the copolymer fractions more water soluble and provide better compatibility in gel and mousse systems. Examples of (meth)acrylates, include but are not limited to, methyl acrylate, ethyl acrylate, butyl acrylate, methyl methacrylate and butyl methacrylate and mixtures thereof.

In the case where the narrow molecular weight range copolymers are applied as a gel, carboxylic acid-containing monomers may interfere with the gelling agents. The most widely used gelling agent is a cross-linked polyacrylic acid, such as carbomer, which is well known to be salt sensitive. In an embodiment, when the copolymer fractions of the present invention comprise carboxylic acid monomers and are used in a gelled formulation, thickening and gelling agents that are non-ionic are used. Examples of non-ionic thickeners are of the cellulosic type, such as the Structure® Cell line of ethylhydroxylethylcellulose thickeners available from Akzo Nobel Surface Chemistry LLC, Bridgewater, N.J. These thickening and gelling polymers are available in a variety of molecular weights and substitution levels.

Narrow molecular weight range copolymers as used herein can be used for hair styling applications, such as in hairspray applications. This can be accomplished by using pressurized systems (e.g., aerosol) or by mechanical systems (e.g., pumps). In the case of pressurized systems, the narrow molecular weight range copolymers should exhibit compatibility with both the solvent used and the one or more propellants. Suitable solvents for use in the present invention include, but are not limited to ethanol, isopropanol, chlorinated or fluorinated hydrocarbons, water or mixtures thereof. Suitable propellants for use in the present invention include, but are not limited to hydrocarbons, fluorinated hydrocarbons, chlorinated hydrocarbons, compressed gas, dimethyl ether and compressed volatile liquids.

When the narrow molecular weight range copolymers are applied using a mechanical pump, the solvent system may be water and the hair styling copolymer fractions are completely soluble in water. The copolymer fractions and the hair fixative formulations containing them must also provide storage stability to prevent clogging the pump spray mechanism and to avoid settling or precipitation of the copolymer fractions.

In conventional polymer preparation processes, the process for preparing such copolymers is through solution polymerization of the monomers. A result of this polymerization is a copolymer having a broad distribution of varying degrees of molecular weights. The copolymer is typically described as having a weight average or number average molecular weight. In contradistinction, the narrow molecular weight range copolymers of the present invention may be prepared as in typical polymerizations, but they are then fractionated and isolated to provide select and specific molecular weight ranges of materials.

Narrow molecular weight range copolymers of the present invention can be prepared by a variety of means. As illustrated elsewhere in this document, fractionating a copolymer having a relatively wide molecular weight range can be accomplished by taking advantage of the copolymers solubility in a mixed solvent system and the broad molecular weight copolymer can be fractionated into narrow molecular weight ranges. Other means of controlling the Mw of the copolymer can also be employed to obtain the desired molecular weight range. One examples of this kind of control would be to prepare the narrow molecular weight range copolymer in a solvent which would cause precipitation of the desired Mw range. Other methods such as careful control over the initiator/monomers addition rates, use of chain transfer reagents and techniques such as living free radical polymerizations could also be employed to prepare the narrow molecular weight range copolymers of this invention. Regardless of how the copolymer is made, enhanced performance in stiffness and low viscosity is predicated by having a narrow Mw range copolymer of 70,000 to 90,000 or less than 55,000 Daltons present in the formulation.

Accordingly, in an embodiment, the one or more narrow molecular weight range copolymers has a weight average molecular weight about 70,000 Daltons or greater as measured by Gel permeation chromatography. In another embodiment, the one or more narrow molecular weight range copolymers has a weight average molecular weight in the range from about 70,000 to about 90,000 Daltons. In another embodiment, the one or more narrow molecular weight range copolymers has a weight average molecular weight in the range from about 75,000 to about 80,000 Daltons. In yet another embodiment, the one or more narrow molecular weight range copolymers has a weight average molecular weight about 55,000 Daltons or less. In yet another embodiment, the one or more narrow molecular weight range copolymers has a weight average molecular weight in the range from about 40,000 to about 55,000 Daltons. In another embodiment, the one or more narrow molecular weight range copolymers has a weight average molecular weight from about 45,000 to about 55,000 Daltons.

In another embodiment, the narrow molecular weight range copolymers suitable for use in the present invention are a combination of narrow molecular weight range copolymers having different average molecular weights. For example, in an embodiment a personal care composition includes one or more first narrow molecular weight range copolymers having a weight average molecular weight in the range 70,000 Daltons or greater, in the range about 70,000 to about 90,000 Daltons or in the range from about 75,000 to about 80,000 Daltons in combination with one or more second narrow molecular weight range copolymers having a weight average molecular weight about 55,000 Daltons or less, in the range from about 40,000 to about 55,000 Daltons or in the range from about 45,000 to about 55,000 Daltons.

In addition to the narrow molecular weight range, the narrow molecular weight range copolymers of the present invention have low viscosity in ethanol. That is, at 5% solids in ethanol, and neutralized 100% with 2-amino-2-methyl-1-propanol, the narrow molecular weight range copolymers have viscosities of about 16 centipoises (cps) or less at room temperature (about 23° C.). In another embodiment, the narrow molecular weight range copolymers have viscosities of from about 5 to about 7 cps at room temperature. In yet another embodiment, the viscosity of the isolated hair styling copolymer fractions at 5% solids in ethanol, neutralized 100% with 2-amino-2-methyl-1-propanol, is from about 5.5 to about 6.0 cps.

In an aspect of the invention, the process for fractionating the starting copolymer into fractions comprises the following general steps. The starting copolymer was dissolved in a solvent. Then, a portion of a non-solvent (i.e. a solvent that the copolymer is not soluble in) was added to precipitate some of the copolymer. The precipitated copolymer fraction was then filtered and collected to isolate it. The filtrate was taken and another portion of the non-solvent was added to precipitate another copolymer fraction, which was then filtered and collected to isolate it. This process was repeated several times to collect more copolymer fractions. The final filtrate was then dried to isolate the last fraction of copolymer.

As defined herein, the narrow molecular weight range copolymers are fractions of a copolymer comprising at least two classes of monomers. These two classes include hydrophobic monomers and carboxylic acid monomers. Other monomers can be incorporated into the copolymer fractions in an amount to provide additional functionality, but are typically incorporated in small amounts, for example, generally about 20% or less of the total copolymer (and thus the copolymer fraction) weight, and in another embodiment about 10% or less and in another embodiment about 5% or less.

The first class of monomers in the starting copolymer of this invention from which the narrow molecular weight range copolymers are obtained are acid-containing monomers. These include any ethylenically unsaturated monomers having a free carboxylic acid group. Some non-limiting examples of these acid-containing monomers are maleic acid, fumaric acid, acrylic acid, methacrylic acid, itaconic acid. In an embodiment of the current invention the copolymer will contain a mixture of one or more acid-containing monomers.

In an embodiment of the invention, the one or more acid-containing monomers will be present in the copolymer fractions at a level of about 5 to about 25 weight percent of the total weight of the dry, un-neutralized copolymer fractions. In another embodiment, the acid monomers will be present in an amount of about 10 to about 20 weight percent of the dry un-neutralized copolymer fractions, and in another embodiment about 12 to about 20 weight percent.

In another embodiment, the copolymer fractions will have an acidity of about 1.5 meq/g to about 3.2 meq/g, as determined by titration with 0.1 N NaOH, where the acidity is reported as the number of milliequivalents of NaOH needed to neutralize one gram of copolymer. In yet another embodiment the copolymer fractions will have an acidity of from about 1.8 meq/g to about 2.6 meq/g as determined by titration with 0.1 N NaOH.

The second class of monomers in the starting copolymer of this invention from which the narrow molecular weight range copolymers are obtained are hydrophobic monomers. This class of monomers are ethylenically unsaturated monomers and are uncharged (i.e. nonionic) and generally have an alkyl group from 1 to about 8 carbon atoms. The monomers can be based on acrylic type monomers, such as methyl methacrylate or ethyl acrylate. These hydrophobic monomers can also be based on acrylamide type monomers such as t-octylacrylamide, butylacrylamide, and methyl acrylamide. In an embodiment of the current invention the copolymer fractions will contain a mixture of one or more hydrophobic monomers.

In an embodiment of the invention, the one or more hydrophobic monomers will be present in the copolymer fractions from about 68 to about 83 weight percent of the dry un-neutralized copolymer fractions. In another embodiment the hydrophobic monomer will be present in copolymer from about 70 to about 80 weight percent of the dry un-neutralized copolymer fraction weight. In another embodiment of the invention the hydrophobic monomer will be chosen from the group consisting of methyl methacrylate, ethyl methacrylate, t-octylacrylamide, ethylacrylamide, t-butylmethacrylate, t-butylacrylamide, and methacrylamide or mixtures thereof.

A third class of optional monomers that may be present in the starting copolymer of this invention from which the narrow molecular weight range copolymers are obtained are hydrophilic monomers. This class of ethylenically unsaturated monomers generally contains monomers with hydroxyl groups or amine groups. Some examples of these monomers are hydroxypropyl methacrylate, t-butylaminoethyl methacrylate. In an embodiment of the current invention the copolymer will contain a mixture of one or more hydrophilic monomers. In an embodiment of the invention, the one or more hydrophilic monomers are present in an amount of about 1 to about 25 weight percent of the dry un-neutralized copolymer fractions and in another embodiment of from about 5 to about 15 weight percent of the dry un-neutralized copolymer fractions.

Some non-limiting examples of the copolymers and copolymer fractions of this invention are: octylacrylamide/acrylates/butylaminoethyl methacrylate copolymer (e.g. Amphomer® resin), Acrylates Copolymer (e.g., Luvimer® Polymer is a terpolymer of t-butyl acrylate/ethyl acrylate and methacrylic acid), a copolymer of VA/Crotonates/Vinyl Neodecanoate Copolymer such as Resyn® 28-2913 (a terpolymer of vinyl acetate, crotonic acid and vinyl neodecanoate) and other commercially available polymers of this type. Amphomer® and Resyn® are available from (Akzo Nobel Personal Care LLC, Bridgewater, N.J. 08559) and Luvimer® polymers are available from BASF Aktiengesellschaft, 67056 Ludwigshafen, Germany). In an embodiment of the present invention the narrow molecular weight range copolymers are acrylates copolymer or octylacrylamide/acrylates/butylaminoethyl methacrylate copolymer or combinations thereof.

Optional polymers can also be added to the hair fixative formulation in addition to the polymer fractions of this invention. Some non-limiting examples are Polyquaternium-11, such as Gafquatt® 734 or Gafquat® 755N; a hydrophilic Polyether Urethane such as Polyurethane Resin 142-89; a Polyquaternium 4 such as Celquat® L-200 or Celquat® H-100; a polyvinylpyrrolidine such as PVP K-60, K-90, K-120 (PVP); a copolymer of PVP/VA such as 335, 535, 735, 630; a copolymer of Acrylate/Acrylamide Copolymer such as Ultrahold; a PVP/Dimethylaminoethyl methacrylate copolymer such as Copolymer 845; and a PVP/VA Copolymer such as E-635, Gantrez® 425, 335, and 215 and a copolymer of PVM/MA Decadiene Crosspolymer such as LoVocryl®

Another desirable property of the copolymer fractions is shampoo removability. Residual copolymer fractions left on the hair after a single wash cycle will lead to unpleasant build-up over time and a sticky feel to the touch. To provide this property the amount of the anionic monomer is controlled with respect to the other monomers and then neutralized after polymerization to generate the carboxylic acid salt(s). Typical neutralizing agents are hydroxides of alkaline and alkaline earth metals, organic amines, carbonate and bicarbonate salts. In an embodiment of this invention the neutralizing agent is chosen from the group consisting of triethanol amine (TEA), 2-amino-2-methyl-1-propanol (AMP), sodium hydroxide, potassium hydroxide and sodium carbonate and mixtures thereof.

For the isolated hair styling copolymer fractions to exhibit shampoo removability, the carboxylic acid functionality must be at least partially neutralized. In an embodiment of this invention the carboxylic acid groups of the copolymer fractions (acid containing monomers described above) will be fully or partially neutralized and in another embodiment, the copolymer fractions may be from about 10 to about 100% neutralized. In another embodiment the acid groups will be neutralized from about 50 to about 95% and in another embodiment the neutralization of the copolymer fractions is from about 75 to about 95%. In yet another embodiment the acid groups will be neutralized from about 75 to about 90%, and in another embodiment about 75% to about 85%. In still another embodiment the acid groups will be neutralized from about 85 to about 95%.

The personal care compositions of the present invention contain an effective amount of the one or more narrow molecular weight range copolymers. In an embodiment of this invention the narrow molecular weight range copolymers will be present from about 0.1 to about 10 weight percent of the total weight of the formulation. In another embodiment the copolymer fractions will be present from about 0.1 to about 0.5 weight percent of the total weight of the formulation. In another embodiment the copolymer fractions can be present at from about 1 to about 3% of the total weight of the formation.

Additionally the hair styling formulations of the invention may contain a solvent for applying the fixative formulation. In an embodiment of this invention, the formulation will contain no more than about 85% of volatile organic compounds (VOC, aka-solvents or propellants), with the remainder of the solvent being water. In another embodiment, the formulation will contain no more than about 55% VOC.

In an aspect, the invention provides a method for producing a hair styling composition comprising dissolving one or more narrow molecular weight range copolymers in the solvent and neutralizing the resultant solution.

In an embodiment of the invention, the narrow molecular weight range copolymers are used in an aerosol formulation, wherein a propellant is present. A skilled artisan will recognize that these propellants will generally add to the overall VOC of the formulation and that the copolymer fraction system must be compatible with the propellant to avoid problems (nozzle clogging, spitting, poor spray pattern, etc.) during the atomization and application to the hair.

Additionally, a variety of adjunct or ancillary ingredients may be added to the hair fixative composition to provide functions other than fixative properties. Examples of these ancillary ingredients are preservatives, colorants, fragrances, viscosity modifiers, vitamins, herbal extracts such as sterols, triterpenes, flavonoids, coumarins, non-glycosidic diterpenes (sterebins) spathulenol, decanoic acid, 8,11,14-ecosatrienoic acid, 2-methyloctadecane, pentacosane, octacosane, stigmasterol, bsitosterol, a- and b-amyrine, lupeol, b-amyrin acetate, and pentacyclic triterpene, include sunscreen actives such as such as a p-methoxycinnamate or an aminobenzoate (UVB absorber) or benzone or an anthranilate (UVA absorber medicaments, moisturizers, anti-itch or anti-dandruff ingredients and the like.

### EXAMPLES

The following examples are intended to exemplify the present invention but are not intended to limit the scope of the invention in any way. The breadth and scope of the invention are to be limited solely by the claims appended hereto.

### Example 1

### Fractionation of AMPHOMER—

To a stirred solution of 800 g of methanol in a 2 L beaker was added 200 g of AMPHOMER® copolymer (available from Akzo Nobel Surface Chemistry LLC, Bridgwaterm N.J.). To the stirred solution was added 150 g of water. The precipitate that formed was filtered though a Buchner funnel and collected as fraction 1, after drying this gave 20 g of Fraction 1. The resulting filtrate was put back into the 2 L beaker. To the stirred solution was added 150 g of water. The precipitate that formed was filtered though a Buchner funnel and collected as fraction 2, after drying this gave 20 g of Fraction 2. The resulting filtrate was put back into the 2 L beaker. To the stirred solution was added 150 g of water. The precipitate that formed was filtered though a Buchner funnel and collected as fraction 3, after drying this gave 10 g of Fraction 3. The resulting filtrate was put back into the 2 L beaker. To the stirred solution was added 150 g of water. The precipitate that formed was filtered though a Buchner funnel and collected as fraction 4, after drying this gave 45 g of Fraction 4. The resulting filtrate was dried to 100 g as fraction 5.

Molecular Weight Determination—

Molecular weight of the copolymer fractions was determined using Gel Permeation Chromatography. N,N-Dimethylacetamide, with 0.03M Sodium Nitrate was used as the mobile phase with a refractive index detector. Narrow range polystyrene standards were used. Both Mw (weight average molecular weight) and Mn (number average molecular weight) were determined for the polymers.

Viscosity Determination—

Anhydrous viscosities were determined by dissolving 3.0 g of polymer in a sufficient amount ethanol to bring the total to 60.0 g. Based on the acidity of the copolymer 100% of the acid groups were neutralized with 2-amino-2-methyl-1-propanol. The viscosity of the 5% solution was then measured at 23° C. using a Brookfield RVT viscometer (obtained from Brookfield Engineering Laboratories of Middleboro, Mass.) equipped with a UL adapter at 100 rpm.

### Preparation of Hairspray Formulations:

### Hair Fixative Formulations

The copolymer fractions were formulated into anhydrous and 55% VOC aerosol hair spray systems according to the following formulations. All values reported are parts by weight, based on the total weight of the hair spray composition.

TABLE 1

| | Parts by Weight Dry Basis | |
|---|---|---|
| Ingredient | Anhydrous | 55% VOC |
| Copolymer | 3.0 | 3.0 |
| Anydrous Ethanol | 56.4 | 22.0 |
| Deionized Water | 0.0 | 41.4 |
| 2-Amino-2-Methyl-1-Propanol | 0.6 | 0.6 |
| Dimethyl Ether | 40.0 | 33.0 |

The copolymer fractions, ethanol, and deionized water were mixed until homogeneous. Solutions were filtered and filled into aerosol containers. Cans were charged with dimethyl ether propellant.

The Anhydrous sprays were delivered with aVX-81 valve (made by Aptar of Cary, Ill.), (no vapor tap*0.013″ stem orifice*0.122″ dip tube diameter) having a VX/XL 200 MISTY, (0.020″ orifice) actuator (made by Aptar of Cary, Ill.).

The 55% VOC sprays were delivered with a VX-81 valve (made by Aptar of Cary Ill.), (0.010″ vapor tap*0.016″ stem orifice*0.122″ dip tube diameter) having a VX/XL200 MISTY (0.020″ orifice) actuator (made by Aptar of Cary Ill.).

Diastron HairSwatch Stiffness—

Stiffness is measured as the amount of work to deflect a hair swatch 10 mm at a rate of 50 mm/min. Stiffness is measured by the following procedure.

Five 6-inch virgin brown hair swatches obtained from International Hair Importers & Products Inc. of Glendale, N.Y. were used for each sample to be tested. To each swatch a 2 second burst aerosol is sprayed to each side of the swatch. The swatches are allowed to dry overnight in a constant temperature constant humidity room maintained at 72F and 50% relative humidity.

The swatches are tested the next day using a Diastron MTT 160 miniature tensile tester from DIA-STRON Limited of Southway, Andover, Hampshire England with a stiffness jig available from the manufacturer of the instrument. Each hair swatch is then laid across two lower horizontal prongs (or bars) separated by 10 cm. The instrument than applies a force to the hair with a 1 cm diameter bar perpendicular to the horizontal swatch and between the two lower bars to bend the swatch a distance of 10 mm. The work, in joules is the stiffness of the hair swatch. For each sample being analyzed this is done for five 6-inch swatches and the average is calculated.

TABLE 2
Viscosity and molecular weight of various copolymer fractions

| | | | | Isolated | % of | |
|---|---|---|---|---|---|---|
| | | | PDI | Dry | original | Anhydrous |
| | Mw | Mn | (Mw/Mn) | Material (g) | 200 g | cps |
| octylacrylamide/acrylates/ | 64,219 | 18,378 | 3.94 | | | 5.2 |
| butylaminoethyl | | | | | | |
| methacrylate copolymer | | | | | | |
| Fraction 1 | 94,251 | 31,451 | 3.00 | 20 | 10.0 | 6.3 |
| Fraction 2 | 77,483 | 27,271 | 2.84 | 20 | 10.0 | 5.6 |
| Fraction 3 | 68,467 | 18,975 | 3.61 | 10 | 5.0 | — |
| Fraction 4 | 69,457 | 24,597 | 2.82 | 45 | 22.5 | 5.4 |
| Fraction 5 | 50,645 | 12,105 | 4.18 | 100 | 50.0 | 4.1 |

For Fraction 3, the amount of material recovered was insufficient make hair sprays of this fraction, thus viscosity was not recorded.

Table 2 shows the relative amount of material and molecular weight of each fraction isolated. Fraction 5 represents the high weight fraction and the most soluble as it did not precipitate from the solvent in the separation process.

TABLE 3
Hair Swatch stiffness of various copolymer fractions

| | Anhydrous |
|---|---|
| | Joules |
| octylacrylamide/acrylates/butylaminoethyl | 0.0060 |
| methacrylate copolymer | |
| Fraction 1 | 0.0061 |
| Fraction 2 | 0.0070 |
| Fraction 3 | — |
| Fraction 4 | 0.0059 |
| Fraction 5 | 0.0073 |

Table 3, shows the stiffness of each fraction of the copolymer and the relation of stiffness to Mw. Surprisingly, Fractions 2 and 5 gives significantly better stiffness yet have lower molecular weight than other fractions (e.g. Fraction 1). While the PDI of Fraction 5 is above 4, it still provides very good stiffness. Without wanting to be bound by theory, it is believed that this is due primarily to the very low molecular weight residuals from the process of making Fraction 5. Based on the above experimental results, it would be expected that if further purification was provided, that the PDI would be significantly lower than 3 and would give even better results. Techniques such as dialysis or column chromatography could be employed to remove the low (less than about 10,000 Daltons) molecular weight residuals from Fraction 5.

In an attempt to determine the roll of the high molecular weight range copolymer, the octylacrylamide/acrylates/butylaminoethyl methacrylate copolymer for dissolved in ethanol and the highest Mw was precipitated and isolated. The remaining copolymer was then recovered from the solvent by evaporation and all three copolymers amples were evaluated for stiffness. The results are shown below.

TABLE 4
Hair Swatch stiffness of various copolymer fractions

| | Anhydrous |
|---|---|
| | Joules |
| octylacrylamide/acrylates/butylaminoethyl | 0.0064 |
| methacrylate copolymer. | |
| Fraction 1 | 0.0063 |
| All other fractions | 0.0049 |

Table 4 shows that while the highest molecular weight fraction gives about the same stiffness as the broad or wide molecular weight range copolymer, the combination of fractions 2 through 4 gave a much lower stiffness than compared to either fraction 2 or fraction 5 alone. This experiment demonstrates the benefit of the narrow molecular weight range copolymer in providing maximum stiffness yet keeping the viscosity of the formulation as low as possible.

All documents cited in the Detailed Description of the Invention are, in relevant part, incorporated herein by reference; the citation of any document is not to be construed as an admission that it is prior art with respect to the present invention.

While particular embodiments of the present invention have been illustrated and described herein, the invention is not intended to be limited to the details shown. Rather, various modifications may be made in the details within the range and scope of equivalents of the claims and without departing from the spirit and scope of the invention.

1. A personal care composition comprising one or more narrow molecular weight range copolymers and one or more solvents, wherein the one or more narrow molecular weight range copolymers has a weight average molecular weight range chosen from 70,000 to 90,000 Daltons or 55,000 Daltons or less and wherein the copolymer is acrylates copolymer or octylacrylamide/acrylates/butylaminoethyl methacrylate copolymer.

2. The personal care composition of claim 1 wherein the one or more narrow molecular weight range copolymers has a weight average molecular weight of from 75,000 to 80,000 Daltons.

3. The personal care composition of claim 1 wherein the one or more narrow molecular weight range copolymers has a viscosity from about 5 to about 7 centipoises, preferably 5.5 to 6.0 cps, in ethanol at 5% solids, at 23° C.

4. The personal care composition of claim 1 wherein the one or more narrow molecular weight range copolymers has been neutralized 100% with 2-amino-2-methyl-1-propanol.

5. The personal care composition of claim 1 wherein the one or more solvents is selected from the group consisting of ethanol, isopropanol, chlorinated or fluorinated hydrocarbons, water and mixtures thereof.

6. The personal care composition of claim 1 wherein the one or more narrow molecular weight range copolymers comprises one or more acid-containing monomers wherein the one or more acid-containing monomers is an ethylenically unsaturated monomer having at least one free carboxylic acid group.

7. The personal care composition of claim 6 wherein the one or more acid-containing monomers is in the narrow molecular weight range copolymers in an amount of 5 to 25 weight percent of the total weight of the dry, un-neutralized narrow molecular weight range copolymers.

8. The personal care composition of claim 1 wherein the one or more narrow molecular weight range copolymers comprises one or more hydrophobic monomers, wherein the one or more hydrophobic monomers is an ethylenically unsaturated monomer having an alkyl group of from 1 to about 8 carbon atoms.

9. The personal care composition of claim 8 wherein the one or more hydrophobic monomers is present in the range from 68 to 83 weight percent of the dry un-neutralized isolated copolymer fraction.

10. The personal care composition of claim 1 wherein the one or more narrow molecular weight range copolymers further comprises one or more hydrophilic monomers, wherein the one or more hydrophilic monomer has a hydroxyl group or an amine group.

11. The personal care composition of claim 10 wherein the one or more hydrophilic monomers is present in an amount of 1 to 25 weight percent of the dry un-neutralized isolated copolymer fraction.

12. The personal care composition of claim 1 wherein the one or more narrow molecular weight range copolymers is present in an amount of 0.1 to 10 weight percent, based on the total amount of the personal care composition.

13. The personal care composition of claim 1 further comprising a neutralizing agent.

14. The personal care composition of claim 1 wherein the one or more narrow molecular weight range copolymers includes carboxylic acid groups that are neutralized from 50 to 95%.

15. The personal care composition of claim 1 wherein the personal care composition is a hair spray and further comprises a propellant.
